(19) United States
(12) Patent Application Publication
(10) Pub. No.: US 2005/0250975 A1
(43) Pub. Date: Nov. 10, 2005

(54) Blood pump with dual inlet passages

(76) Inventors: Michel Carrier, Montreal (CA); Andre Garon, Ville d'Anjou (CA); Ricardo Camarero, Verdun (CA); Conrad Pelletier, Montreal (CA); Victor Obeid, Collegeville, PA (US)

Correspondence Address: QUARLES & BRADY LLP, 411 E. WISCONSIN AVENUE, SUITE 2040, MILWAUKEE, WI, 53202-4497, US

(21) Appl. No.: 11/116,750

(22) Filed: Apr. 28, 2005

(30) Foreign Application Priority Data
May 13, 2003 (CA) 2,428,741

(51) Int. Cl. A61N 1/362

(52) U.S. Cl. 600/16

(57) ABSTRACT

The blood pump comprises a stationary housing structure, a rotative impeller, a first inlet and a second inlet. The stationary housing structure has a proximal end and a distal end, and is substantially symmetrical about a longitudinal axis. The rotative impeller is mounted within the stationary housing structure to circulate blood in a blood flow direction extending from the proximal end to the distal end. The first inlet is provided near the proximal end, and leads to a first passage. The second inlet leads to a second passage defining an acute angle with the longitudinal axis. The first and second passages join into a common passage between the second inlet and the rotative impeller. When the rotative impeller is activated, a first predetermined volume of blood flows in the stationary housing structure through the first inlet and a second predetermined volume of blood flows in the stationary housing structure through the second inlet, the second predetermined volume being greater than the first predetermined volume.

### CROSS-REFERENCE TO RELATED APPLICATION

This application is a divisional of international application PCT/CA2004/0074 filed May 13, 2004, which claims priority to Canadian application 2,428,741 filed May 13, 2003.

### STATEMENT REGARDING FEDERALLY SPONSORED RESEARCH OR DEVELOPMENT

### FIELD OF THE INVENTION

The present invention relates to a blood pump comprising a pair of inlet blood passages.

### BACKGROUND OF THE INVENTION

In North America, heart related diseases are still the leading cause of death. Among the causes of heart mortality are congestive heart failure, cardiomyopathy and cardiogenic shock.

The incidence of congestive heart failure increases dramatically for people over 45 years of age. In addition, a large part of the population in North America is now entering this age group. Thus, patients who will need treatment for these types of diseases comprise a larger segment of the population. Many complications related to congestive heart failure, including death, could be avoided and many years added to these patients' lives if proper treatments were available.

The types of treatments available for patients experiencing heart failure depend on the extent and severity of the illness. Many patients can be cured with rest and drug therapy but there are still severe cases that require various heart surgeries, including heart transplantation. Actually, the mortality rate for patients with cardiomyopathy who receive drug therapy is about 25% within two years and there still is some form of these diseases that cannot be treated medically. One of the last options that remain for these patients is heart transplantation. Unfortunately, according to the procurement agency UNOS (United Network for Organ Sharing in the United States), the waiting list for heart transplantation grows at a rate of more than twice the number of heart donors.

Considering the above facts, it appears imperative to offer alternative treatments to heart transplantation. The treatment should not only add to a patient's longevity but also improve his quality of life. In this context, mechanical circulatory support through Ventricular Assist Devices (VAD) is a worthwhile alternative given the large deficiency in the number of available organ donors. It is estimated that eight thousand (8,000) patients per year in Canada and seventy-six thousand patients (76,000) per year in the United States could benefit from VADs.

In 1980, the National Heart, Lung and Blood Institute (NHLBI) of the United States defined the characteristics for an implantable VAD (Altieri, F. O. and Watson, J. T, 1987, “Implantable Ventricular Assist Systems”, Artif Organs, Vol. 11, pp. 237-246). These characteristics include medical requirements including restoration of hemodynamic function (pressure and cardiac index), avoidance of hemolysis, prevention of clot formation, infection and bleeding, and minimisation of the anti-coagulation requirement. Further technical requirements include: small size, control mode, long life span (>2 years), low heating, noise and vibration.

Several VADs have been developed to enhance blood circulation and reduce the load on the heart of patients having poor hemodynamic functions (low cardiac output, low ejection fraction, low systolic pressure). These VADs include pulsatile and non-pulsatile VADs.

A first example of non-pulsative VADs are radial-flow blood pumps. In radial-flow blood pumps, the rotation of the impeller produces a centrifugal force that drags blood from the inlet port to the outlet port. A problem related to radial-flow blood pumps is that although they are much smaller than pulsatile VADs, they are still too large to be totally implanted in a human thorax thus eliminating any intra-ventricular implantation.

A second example of non-pulsative VADs are axial-flow blood pumps. These axial-flow blood pumps decrease the hemolysis rate by decreasing the time of exposure of the blood to friction forces and by reducing the intensity of these forces. Another interesting advantage is that axial-flow blood pumps are generally much smaller than radial-flow blood pumps, and can be much more easily implanted in the human body, even in the left ventricle of the heart, for medium and long term mechanical cardiac support.

Although the above-described VADs can achieve the goals of restoring the hemodynamic functions and improving end organ perfusion, both power and pumping efficiency of these VADs can still be improved. Also, hemolysis and thrombus formation are still important problems requiring investigation.

### SUMMARY OF THE INVENTION

In order to improve VADs, the present invention is concerned with a blood pump comprising: a stationary housing structure having a proximal end and a distal end, this stationary housing structure being substantially symmetrical about a longitudinal axis; a rotative impeller mounted within the stationary housing structure to circulate blood in a blood flow direction extending from the proximal end to the distal end; a first inlet provided near the proximal end, this first inlet leading to a first passage; and a second inlet leading to a second passage defining an acute angle with the longitudinal axis. The first and second passages join into a common passage between the second inlet and the rotative impeller. When the rotative impeller is activated a first predetermined volume of blood flows in the stationary housing structure through the first inlet and a second predetermined volume of blood flows in the stationary housing structure through the second inlet, wherein the second predetermined volume is greater than the first predetermined volume.

According to a non-restrictive illustrative example, the second predetermined volume of blood entering the stationary housing structure is about three times greater than the first predetermined volume of blood entering this stationary housing structure.

The foregoing and other objects, advantages and features of the present invention will become more apparent upon reading of the following non-restrictive description of illustrative embodiments thereof, given by way of example only with reference to the accompanying drawings.

### BRIEF DESCRIPTION OF THE DRAWINGS

In the appended drawings:

FIG. 1 is a cross sectional view of a human heart in which a non-restrictive, illustrative intra-ventricular embodiment of a mixed-flow blood pump according to the present invention is implanted;

FIG. 2 is a graph showing, for different types of pumps, a curve relating a specific pump rotation speed Nₛ to a specific pump diameter Dₛ at the points where the pump is operating at maximum hydraulic efficiency;

FIG. 3 is a side elevational, cross sectional view of the intra-ventricular mixed-flow blood pump of FIG. 1;

FIG. 4 is a perspective view of radial blades of an outflow stator of the intra-ventricular mixed-flow blood pump of FIG. 3, showing an example of configuration of these radial blades;

FIG. 5 is a rear perspective view of a frusto-conical inflow bushing of the intra-ventricular mixed-flow blood pump of FIG. 3, showing the configuration of the inner face of this bushing;

FIG. 6 is a side perspective view of the frusto-conical inflow bushing of FIG. 5;

FIG. 7 is a front perspective view of the frusto-conical inflow bushing of FIGS. 5 and 6;

FIG. 8 is a front perspective view of a frusto-conical outflow bushing of the intra-ventricular mixed-flow blood pump of FIG. 3, showing the configuration of the inner face of that outflow bushing;

FIG. 9 is a perspective view of an impeller blade structure of the intra-ventricular mixed-flow blood pump of FIG. 3, comprising an annular member on which a set of impeller blades are mounted;

FIG. 10 is a side elevational view of a portion of an impeller drive shaft of the intra-ventricular mixed flow blood pump of FIG. 3 and of the annular member of the impeller blade structure of FIG. 9, showing how the latter annular member is mounted on the impeller drive shaft;

FIG. 11 is side elevational view of an impeller blade of the impeller blade structure of FIG. 9, showing details of structure of this impeller blade;

FIG. 12 is a side elevational and cross sectional view of an illustrative extra-ventricular embodiment of the mixed-flow blood pump according to the present invention, incorporating the elements and/or structure shown in FIGS. 4-11; and

FIG. 13 is a schematic view of an illustrative embodiment of a VAD system implanted in a human being and comprising the mixed-flow blood pump of FIG. 3.

### DESCRIPTION OF THE ILLUSTRATIVE EMBODIMENTS

The non-restrictive illustrative embodiments of the present invention will be described in connection with a mixed-flow blood pump that can be used as part of:
an intra-ventricular VAD;
an extra-ventricular VAD, for example a VAD located in a patient's abdomen or thorax; or
an extra-corporal VAD, for example in a bridge to heart transplantation.
   It should also be understood that the mixed-flow blood pump can be used either in temporary VADs, or medium and long term VADs.

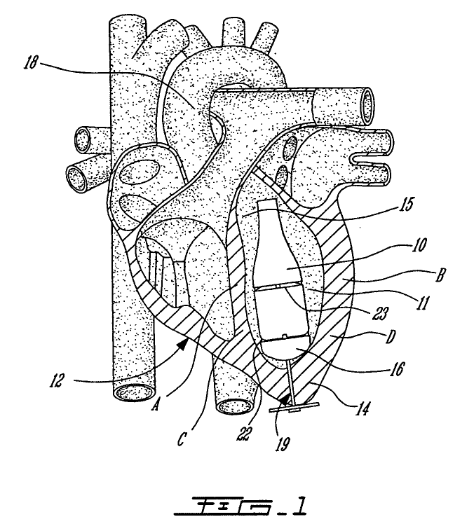

FIG. 1 illustrates a possible position for an illustrative intra-ventricular embodiment 10 of the mixed-flow blood pump in the left ventricle 11 of a patient's heart 12.

The intra-ventricular mixed-flow blood pump 10 has been designed and dimensioned to fit in small adults and in teens. Feigenbaum, Harvey, “Echocardiography”, 5th Edition, 1994, Lea & Febiger, Philadelphia, has determined that for 95% of the population the internal diameter of the left ventricle 11 ranges from 37 to 46 mm in diastole and between 22 to 31 mm in systole. This diameter is determined at the centre of the ventricular length (segment AB of FIG. 1). The diameter near the apex at the first third of the ventricular length is about 15 mm (segment CD of FIG. 1). The internal length of the ventricle from the apex to the aortic valve ranges from 55 to 70 mm. Finally, the other important parameter is the surface of the aortic valve opening, which ranges from 2.5 to 4 cm².

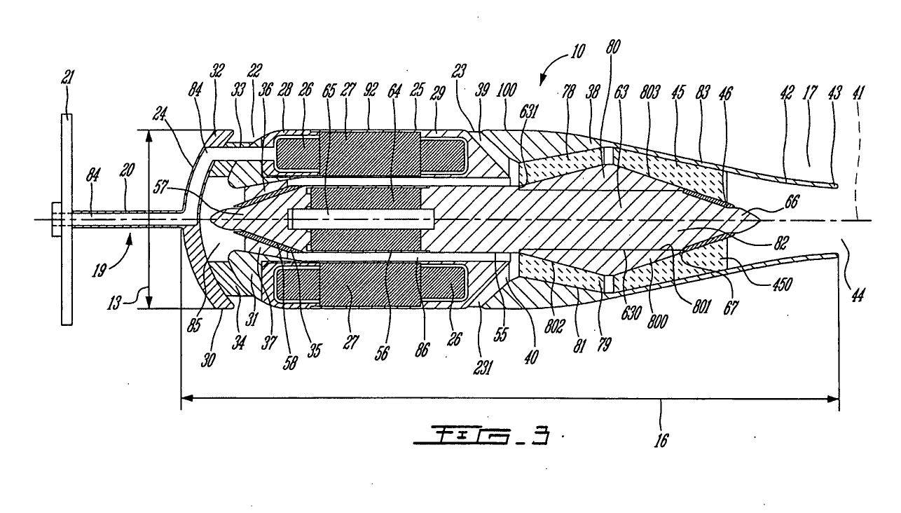

Obviously, the external design (shape and size) of the intra-ventricular mixed-flow blood pump 10 (FIG. 1) will depend on the above anatomic dimensions of the left ventricle 11. FIG. 3 shows the external outline of the intra-ventricular mixed-flow blood pump 10. The diameter of the intra-ventricular mixed-flow blood pump 10 constitutes a compromise between pumping requirements and minimal interference with heart contraction. In the intra-ventricular mixed-flow blood pump 10, the maximum allowable diameter 13 (FIG. 3) is about 22 mm, which is the diameter of the left ventricle 11 in systole. This dimension is reasonable since people with heart failure generally have dilated ventricles.

The maximum length of the intra-ventricular mixed-flow blood pump 10, as illustrated in FIG. 3, is set in regard of the average distance between the apex 14 and the aortic valve 15 of the heart 12. The length 16 (FIG. 3) of the mixed-flow blood pump 10 is about 65 mm.

It should be understood that the size and shape of the intra-ventricular mixed-flow blood pump 10 could also be adapted to meet the anatomical dimensions of individuals falling outside the above described 95% of the population. Similarly, the size and shape could be adapted to specific and particular individuals and heart conditions.

Since the intra-ventricular mixed-flow blood pump 10 will be totally immersed inside the left ventricle 11, blood will circulate around the pump 10. As a consequence, the external surfaces of the intra-ventricular mixed-flow blood pump 10 will be as smooth as possible and avoid as much as possible abrupt deviations to thereby minimise recirculation and stagnation zones that can be at the origin of clot formation. To overcome this problem, the intra-ventricular mixed-flow blood pump 10 may be machined, for example, from surgical quality titanium.

From a surgical point of view, a non-limitative illustrative procedure for inserting the intra-ventricular mixed-flow blood pump 10 is to use the same approach as with cardiac valve replacement. According to this procedure, an incision is made at the root of the aorta 18 (FIG. 1) and the pump 10 is inserted though the aortic valve and then into the left ventricle 11. The mixed-flow blood pump 10 is then pushed until its base reaches the myocardium at the apex 14 and then fixed in place.

To prevent motion thereof, the intra-ventricular mixed-flow blood pump 10 is finally fixed by means of a fixation mechanism 19 (FIGS. 1 and 3) provided at the inflow end of the pump 10. As a non-limitative example, the fixation mechanism 19 (FIG. 3) comprises:
an elongated hollow needle 20 projecting axially from the inflow end of the pump 10, this needle 20 being driven from the inside of the left ventricle 11 through the myocardium and the epicardium at the apex 14 of the heart 12; and
a fixation disk 21 fastened to the free end of the needle 20 on the outside of the heart 12 to firmly fasten the mixed-flow blood pump 10 within the left ventricle 11.

As a non-limitative example, both the free end of the needle 20 and the fixation disk 21 will be threaded to allow the fixation disk 21 to be screwed on the free end of the elongated hollow needle 20. Further rotation of the fixation disk 21 on the needle 20 will then be prevented by any suitable means.

Of course, it is within the scope of the present invention to use any suitable fixation mechanism other than the needle 20 and disk 21.

Since one of the main functions of the intra-ventricular mixed-flow blood pump 10 is to restore the hemodynamic function in patients with cardiac failure, and depending on the severity of the failure and the BSA (Body Surface Area), the intra-ventricular mixed-flow blood pump 10 is susceptible to work at flow rates between 2 to 6 litres per minute (l/min) against a pressure as high as 120 mmHg and, more commonly, at a flow rate between 3 to 5 l/min against a pressure of 80 mmHg. A high efficiency pump design is therefore required.

When designing turbine pumps, dimensionless characteristic values are used to compare different pump configurations. Dimensionless characteristic values provide useful indications to pump designers of expected performance regardless of the size of the pump, a comparison which would otherwise prove difficult given a virtually infinite number of operating parameters that depend on infinite variations of internal pump geometry. These dimensionless characteristic values, therefore, can be used to provide an objective starting point for the selection of a general pump configuration.

Two of these dimensionless characteristic values are the specific rotation speed Nₛ of the pump and the specific pump diameter Dₛ. They are defined as follows:

$$\begin{matrix}{N}_{s} = \frac{Ω {Q}^{1 / 2}}{{H}^{3 / 4}} & ( 1 ) \\ {D}_{s} = \frac{D \cdot {H}^{1 / 4}}{{Q}^{1 / 2}} & ( 2 )\end{matrix}$$

where Ω is the speed of rotation of the pump 10 in radians/second, Q is the flow rate in m³/second, H is the head (i.e. the gain in pressure) of the pump 10 and D the diameter of the pump, both in meters. Nₛ remains the same regardless of the size of the pump and therefore provides an accurate measure of the performance of a given pump design. Dₛ relates the pump diameter to the pump head H and flow rate Q.

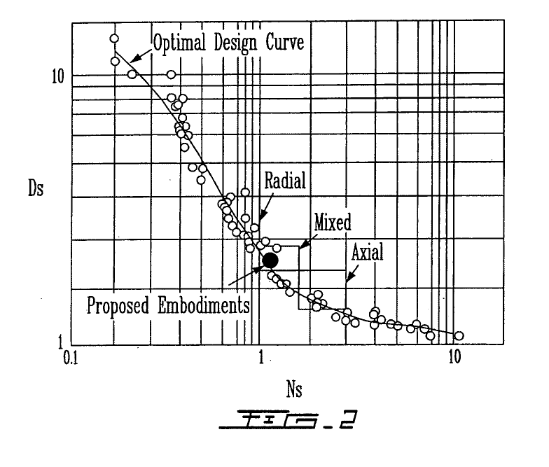

Referring to FIG. 2, the design curve relates the specific speed Nₛ with the specific diameter Dₛ to yield the optimal pump configuration. Specifically, if the configuration of Nₛ and Dₛ falls on the curve, the maximum hydraulic efficiency of the design is greater than if it falls away from the curve. In this regard, hydraulic efficiency is expressed as the percentage of the power input to the pump which is converted to energy of movement of the fluid within the pump. From the curve of FIG. 2 and equation (1) above, it follows that optimally efficient pumps having a higher specific speed also have a smaller size.

In order to determine an optimised choice for a pump, it is necessary to evaluate the specific speed Nₛ in light of the characteristics in terms of head H and flow rate Q projected for the pump. As discussed above, the pump will typically be operated with a flow rate of 5 litres/minutes and a head of approximately 100 mmHg. Additionally, current motor technology provides small yet efficient motors operating at a speed of 7,500 RPM. This gives a specific speed Nₛ of 1.12 and a specific diameter Dₛ of 2.45 for a maximum internal diameter of 12 mm.

Still referring to FIG. 2, an indication is given to the ranges of Nₛ and Dₛ within which a given pump configuration will provide efficient operation. The specific speed Nₛ of 1.12 falls within a transition region of the curve between axial-flow and radial-flow pumps. In this transition region, a mixed-flow pump topology would yield a higher efficiency than purely radial-flow or axial-flow pumps. Additionally, the specific diameter Dₛ is around 2.45 which, by applying Equation (2) above, yields a maximum impeller drive shaft diameter of 12 mm, i.e. a very small pump. For these reasons, a mixed-flow pump design was selected for the intra-ventricular pump 10.

The structure and operation of the non-restrictive illustrative embodiment 10 of the intra-ventricular mixed-flow blood pump will now be described.

Referring to FIG. 1, the intra-ventricular mixed-flow blood pump 10 rests on the bottom of the left ventricle 11, in the region of the apex 14 of the heart 12.

As shown in FIGS. 1 and 3, in order to prevent the inner walls of the left ventricle 11 from completely obstructing blood intake, the intra-ventricular mixed-flow blood pump 10 comprises a stationary housing structure 100 (FIG. 3) including two axially spaced apart, annular radial-flow inlets 22 and 23. Additionally, the inflow end of the stationary housing structure 100 presents a surface 24 presenting the general configuration of a circular portion of a hemisphere. The diameter of the circular hemisphere portion is set to approximately 20 mm, which is smaller than the segment CD (see FIG. 1) and suitable to reduce the level of pressure on the walls of the left ventricle 11 near the apex 14.

The stationary housing structure 100 of the intra-ventricular mixed-flow blood pump 10 comprises a hollow cylindrical member 25 containing the stator windings such as 26 and the associated magnetic cores such as 27. The hollow cylindrical member 25 is made of two mutually mating annular pieces 28 and 29 to enable insertion of the stator windings 26 and cores 27 within the hollow cylindrical member 25. For example, both the annular pieces 28 and 29 will be threaded to allow said annular pieces 28 and 29 to be screwed on each other. Further rotation of the annular pieces 28 and 29 on each other will then be prevented by any suitable means. Alternatively, the annular pieces 28 and 29 can be laser welded to each other.

The stationary housing structure 100 further comprises an inflow bushing mount 30 mounted on a proximal end of the cylindrical member 25. More specifically, the inflow bushing mount 30 comprises an annular portion 31 profiled to fit on the proximal end of the hollow cylindrical member 25 while defining with this cylindrical member 25 a smooth surface of the annular radial-flow inlet 22. The inflow bushing mount 30 also comprises a wall 32 presenting the general configuration of a circular portion of a hemisphere; the outer face of the hemispheric wall 32 defines the above-mentioned hemispheric surface 24. The inner face of the hemispheric wall 32 is connected to the annular portion 31 through a series of radial blades such as 33 and 34 spread out evenly around a longitudinal axis 41 of the intra-ventricular mixed-flow blood pump 10, within the radial-flow inlet 22. Another function of the radial blades such as 33 and 34 is to straighten out the flow of blood through the radial-flow inlet 22.

An inflow bushing 35 having the general configuration of a frustum of cone is mounted inside the annular portion 31 of the bushing mount 30 and is centered on the longitudinal axis 41 of the blood pump 10. More specifically, the frusto-conical bushing 35 is mounted to the annular portion 31 through a series of radial blades such as 36 and 37 spread out evenly around the axis of the intra-ventricular mixed-flow blood pump 10, more specifically around the frusto-conical bushing 35. As illustrated in FIG. 3, the frusto-conical bushing 35 has an end of larger diameter facing toward the inflow end of the intra-ventricular mixed-flow blood pump 10. Again, another function of the blades such as 36 and 37 is to straighten out the flow of blood passing between the frusto-conical bushing 35 and the annular portion 31 of the inflow bushing mount 30.

The stationary housing structure 100 of the intra-ventricular mixed-flow blood pump 10 further comprises an impeller housing 38 and an outflow cannula 42.

The proximal end of the impeller housing 38 is connected to the distal end of the cylindrical member 25 through a series of radial blades such as 39 and 40 spread out evenly around the longitudinal axis 41 to define the second annular radial-flow inlet 23 between the distal end of the cylindrical member 25 and the proximal end of the impeller housing 38. Another function of the radial blades such as 39 and 40 is to straighten out the flow of blood through the annular radial-flow inlet 23.

As mentioned hereinabove, the first annular radial-flow inlet 22 is axially spaced apart from the second annular radial-flow inlet 23 to reduce as much as possible the effect occlusion of one of the inlets 22 or 23 may have on normal operation of the blood pump 10.

Referring to FIGS. 1 and 3, the diameter of the outflow cannula 42 reduces from the impeller housing 38 to the free end of the outflow cannula to reduce as much as possible the obstruction caused by the intra-ventricular mixed-flow blood pump 10 to the operation of the aortic valve (not shown); since the function of the intra-ventricular mixed-flow blood pump 10 is to assist blood circulation, blood flow contribution from the natural contraction of the heart 12 should be maintained. In the intra-ventricular mixed-flow blood pump 10, the area of the outflow cannula 42, corresponding to diameter 43, is 0.463 cm².

As illustrated in FIG. 3, the impeller housing 38 and outflow cannula 42 are respectively made of two separate, mutually mating pieces in order to enable insertion of the impeller within the impeller housing 38. For example, both the impeller housing 38 and the outflow cannula 42 will be threaded to allow said housing 38 and cannula 42 to be screwed on each other. Further rotation of the impeller housing 38 and outflow cannula 42 on each other will then be prevented by any suitable means. Alternatively, the impeller housing 38 and the outflow cannula 42 can be laser welded to each other.

A blood diffuser (not shown) can be mounted on the free end of the outflow cannula 42 (outflow end of the stationary housing structure 100). The function of the blood diffuser would be to reduce the shear stress on blood cells. Without diffuser, the velocity of blood ejected from the intra-ventricular mixed-flow blood pump 10 is higher than the velocity of blood ejected through the aortic valve 15 of the heart 12. The difference of velocity between the two blood flows would result in shear stresses proportional to this difference. Since the velocity is inversely proportional to the cross-sectional area, a solution for reducing the relative velocity of the blood flows from the pump 10 and from the heart 12 is (a) to increase the area of the orifice 44 of the outflow cannula 42 to thereby reduce the velocity of the flow of blood from the pump 10, and (b) to decrease the area occupied by the blood flow from the heart 12 to increase the velocity of the latter blood flow. This would be exactly the role of the blood diffuser. Of course, parameters such as the angle of opening and the length of the blood diffuser could be adjusted at will to fit the mechanical characteristics of the intra-ventricular mixed-flow blood pump 10 in view of minimising the shear stress on the blood cells.

The outflow cannula 42 comprises an outflow stator 45 formed of a series of inner radial blades spread out evenly around the longitudinal axis 41 and connected to the inner face of the outflow stator 45 by diffusion bound. FIG. 4 illustrates an example of configuration of the radial blades such as 450 of the outflow stator 45. More specifically, the radial blades 450 are configured to straighten out the flow of blood exiting the cannula 42 and are fixedly secured to the inner face of the outflow cannula 42.

An outflow bushing 46 having the general configuration of a frustum of cone is mounted inside the outflow stator 45 and is centered on the longitudinal axis 41. More specifically, the frusto-conical bushing 46 is secured to the radial blades 450 of the outflow stator 45. Finally, the end of larger diameter of the frusto-conical bushing 46 is facing toward the inflow end of the intra-ventricular mixed-flow blood pump 10.

The intra-ventricular mixed-flow blood pump 10 also comprises a rotative impeller 56 provided with an impeller drive shaft 55 centered on the longitudinal axis 41. The impeller drive shaft 55 comprises an inflow end portion 57 formed with an inflow frusto-conical face 58 structured to snugly fit into the inflow frusto-conical bushing 35.

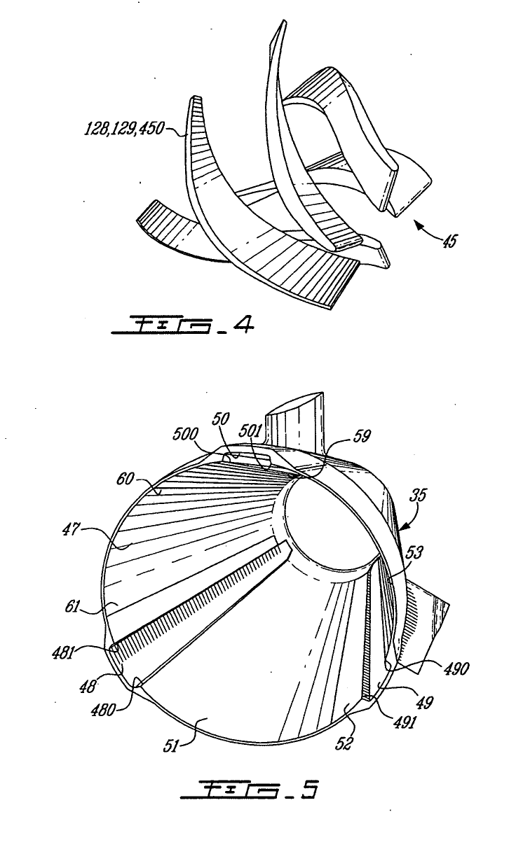

FIG. 5 illustrates the inner face 47 of the inflow bushing 35. Inner face 47 comprises at least three axial grooves 48, 49 and 50 generally rectangular in cross section and evenly spread out around the longitudinal axis 41 (FIG. 3).

As a non-limitative example, from groove 48 to groove 49, the inner face 47 of the inflow bushing 35 successively defines in the direction of rotation of the rotative impeller 56 a taper 51 and a land 52. Taper 51 has a diameter that gradually decreases from groove 48 to land 52, and land 52 has a constant diameter. The land 52 spans an angular sector of approximately 17.5° about the longitudinal axis 41 and presents a clearance of approximately 0.0116 mm to the frusto-conical face 58 (FIG. 3) of the inflow end portion 57 of the impeller drive shaft 55. The taper 51 spans an angular sector of approximately 82.5° about the longitudinal axis 41 and creates, from the groove 48 to the land 52, a gradual 0.030 mm clearance increase. That means that, at the edge 480 separating the taper 51 from the groove 48, there is a clearance of approximately 0.0416 mm. The groove 48 spans an angular sector of approximately 20° about the longitudinal axis 41 computed from the edge 481 joining the land 61 to the groove 48 up to the edge 480 joining the same groove 48 to the taper 51. This 20° angular sector includes the round of edge 481 blending the land 61 with the groove 48 and the round of edge 480 blending the taper 51 with the groove 48.

As a non-limitative example, from groove 49 to groove 50, the inner face 47 of the inflow bushing 35 successively defines in the direction of rotation of the rotative impeller 56 a taper 53 and a land 59. Taper 53 has a diameter that gradually decreases from groove 49 to land 59, and land 59 has the same constant diameter as land 52. The land 59 spans an angular sector of approximately 17.5° about the longitudinal axis 41 and presents a clearance of approximately 0.0116 mm to the frusto-conical face 58 (FIG. 3) of the inflow end portion 57 of the impeller drive shaft 55. The taper 53 spans an angular sector of approximately 82.5° about the longitudinal axis 41 and creates, from the groove 49 to the land 59, a gradual 0.030 mm clearance increase. That means that, at the edge 490 separating the taper 53 from the groove 49, there is a clearance of approximately 0.0416 mm. The groove 49 spans an angular sector of approximately 20° about the longitudinal axis 41 computed from the edge 491 joining the land 52 to the groove 49 up to the edge 490 joining the same groove 49 to the taper 53. This 20° angular sector includes the round of edge 491 blending the land 52 with the groove 49 and the round of edge 490 blending the taper 53 with the groove 49.

As a non-limitative example, from groove 50 to groove 48, the inner face 47 of the inflow bushing 35 successively defines in the direction of rotation of the rotative impeller 56 a taper 60 and a land 61. Taper 60 has a diameter that gradually decreases from groove 50 to land 61, and land 61 has the same constant diameter as the lands 52 and 59. The land 61 spans an angular sector of approximately 17.5° about the longitudinal axis 41 and presents a clearance of approximately 0.0116 mm to the frusto-conical face 58 (FIG. 3) of the inflow end portion 57 of the impeller drive shaft 55. The taper 60 spans an angular sector of approximately 82.5° about the longitudinal axis 41 and creates, from the groove 50 to the land 61, a gradual 0.030 mm clearance increase. That means that, at the edge 500 separating the taper 60 from the groove 50, there is a clearance of approximately 0.0416 mm. The groove 50 spans an angular sector of approximately 20° about the longitudinal axis 41 computed from the edge 501 joining the land 59 to the groove 50 up to the edge 500 joining the same groove 50 to the taper 60. This 20° angular sector includes the round of edge 501 blending the land 59 with the groove 50 and the round of edge 500 blending the taper 60 with the groove 50.

In an example of construction, there are three blades such as 36 and 37 (FIG. 3) aligned with the three grooves 48, 49 and 50, respectively, to minimize blood flow perturbation. A number of blades and grooves different from 3 could obviously be used. Also, the blades such as 36 and 37 are connected to the grooves 48, 49 and 50, respectively, by diffusion bound.

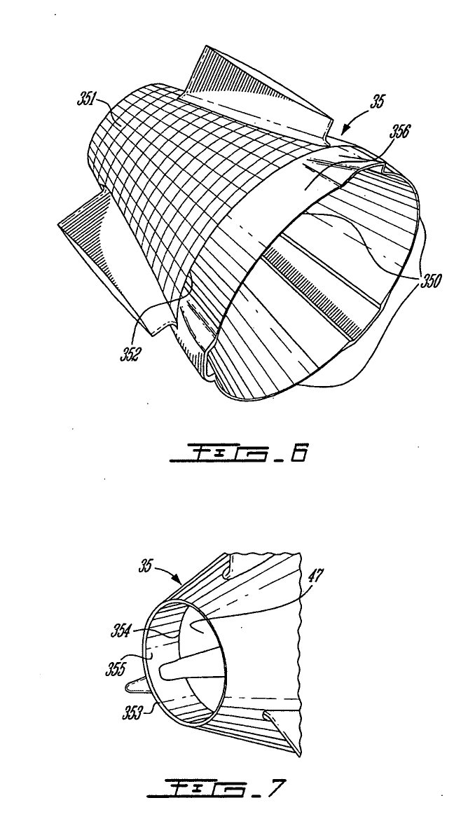

Referring to FIG. 6, the geometry of the curvature of the three trailing edges 350 of the inflow bushing 35 is constant for the three taper/land zones 51/52, 53/59 and 60/61. More specifically, the trailing edges 350 each correspond to an arc of circle of given diameter.

To keep the geometry of the curvature constant in the taper zones 51, 53 and 60, a portion of the outer surface of the inflow bushing 35 has been modified. More specifically, the portion 351 of the outer surface of the inflow bushing 35, shown in meshed area in FIG. 6, runs all around the inflow bushing 35 and is a perfect revolved surface portion. The “perfect circle” 352 outlined in FIG. 6 represents the last section of the inflow bushing 35 where the outer surface is obtained by a revolved feature.

The outer surface of the inflow bushing 35 defines a blended surface portion 356 blending the perfect revolved surface portion with the growth of the taper diameter, to allow the geometry of the curvature of the trailing edges 350 to remain constant while the radial position of each trailing edge 350 changes at the same rate as the taper diameter.

At the leading end 353 of the inflow bushing 35, the annular edge 354 that is created at the intersection between the cylindrical inner surface portion 355 and the inner surface 47 of the inflow bushing 35 including the grooves 48, 49 and 50, the tapers 51, 53 and 60, and the lands 52, 59 and 61 is polished to smoothen that edge 354 (see FIG. 7).

In operation, the lands 52, 59 and 61 form a seat for the inflow frusto-conical face 58 of the inflow end portion 57 of the impeller drive shaft 55. The grooves 48, 49 and 50 enable flow of blood between the faces 47 and 58. The hydrodynamic forces produced by rotation of the impeller drive shaft 55 will produce a thicker film of blood flowing between the frusto-conical face 58 of the inflow end portion 57 of the impeller drive shaft 55 and the tapers 51, 53 and 60, and a thinner film of blood flowing between face 58 and the lands 52, 59 and 61 to thereby lubricate the resulting bearing (frusto-conical face 58 and inner face of the frusto-conical bushing 35). Since blood flows through the gap between the frusto-conical faces 47 and 58, minimal hemolysis, thrombus and clot formation will be produced.

As a non-limitative example, the resulting inflow bearing (frusto-conical face 47 and frusto-conical bushing 35) present the following approximate dimensions and characteristics:
Larger diameter: 8 mm
Smaller diameter: 3 mm
Cone angle: 20°
Axial length: 6.87 mm
Cone length: 7.31 mm
Number of pads (taper and land): 3
Pad angle about the longitudinal axis: 100°
Groove angle about the longitudinal axis: 20°
Taper angle about the longitudinal axis: 82.5°
Taper gradual clearance increase: 0.030 mm
Land angle about the longitudinal axis: 17.5°

Land clearance: 0.0116 mm.

INFLOW 6807 RPM

| C = | Radial | Axial | Axial | Minimum | Power | Maximum |
|---|---|---|---|---|---|---|
| Clearance | Gap | Gap | Load | Film | Loss | Pressure |
| (mm) | (mm) | (mm) | (N) | (mm) | (Watt) | (Pa) |
| 0.01116 | 0.0123 | 0.0339 | 1.01 | 0.0116 | 0.1199 | 74871 |

With a 0.8 N magnetic pull axially at 5 l/min against a pressure of 80 mmHg.

Referring back to FIG. 3, the impeller drive shaft 55 also comprises an outflow portion 63 which, when assembled to the inflow end portion 57 defines a cavity in which a cylindrical permanent magnet 64 is inserted. The windings 26 and the permanent magnet 64 form an electric motor structure operative to set the impeller 56 into rotation; the magnetic field produced by the windings 26 is applied to the magnetic field produced by the permanent magnet 64 to produce a reaction that will set the impeller 56 into rotation.

An axial magnetic pull is produced by slightly, axially offsetting the permanent magnet 64 toward the inflow bushing 35 with respect of the magnetic windings 26. This will produce an axial magnetic pull of the order of, for example, 0,8 N toward the outflow bushing 46, i.e. in a direction opposite to an axial force produced on the impeller drive shaft 55 upon pumping blood.

An axial screw 65 passes through the magnet 64 and screws into both the inflow 57 and outflow 63 portions of the impeller drive shaft 55 to firmly secure these two drive shaft portions 57 and 63 together.

The outflow portion 63 of the impeller drive shaft 55 comprises an outflow end 66 formed with an outflow frusto-conical face 67 structured to snugly fit into the frusto-conical bushing 46.

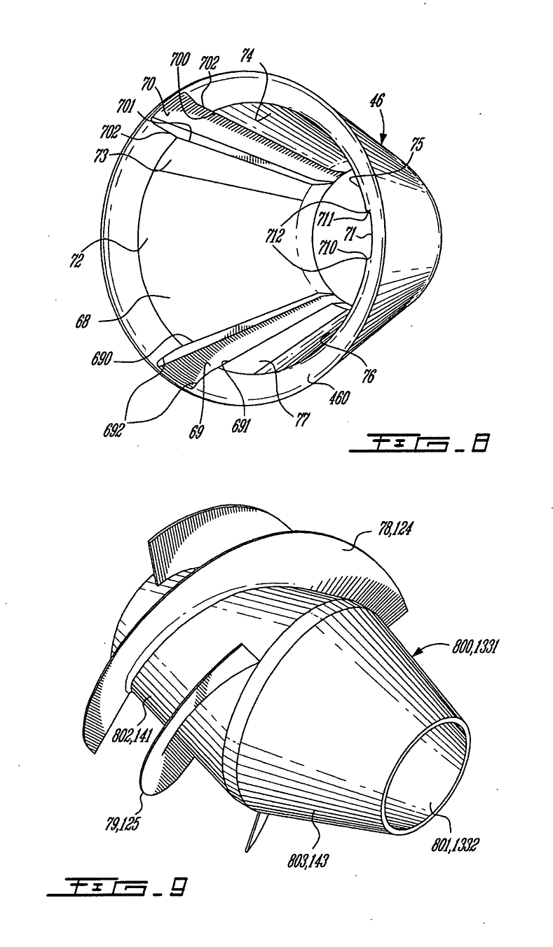

FIG. 8 illustrates the inner face 68 of the outflow bushing 46. Inner face 68 comprises at least three axial grooves 69, 70 and 71 generally rectangular in cross section and evenly spread out around the longitudinal axis 41 (FIG. 3).

As a non limitative example, from groove 69 to groove 70, the inner face 68 successively defines in the direction of rotation of the rotative impleller 133 a taper 72 and a land 73. Taper 72 has a diameter that gradually decreases from groove 69 to land 73, and land 73 has a constant diameter. The land 73 spans an angular sector of approximately 20° about the longitudinal axis 107 and presents a clearance of approximately 0.0226 mm to the frusto-conical face 67 of the outflow portion 63 of the impeller drive shaft 55. The taper 72 spans an angular sector of approximately 80° about the longitudinal axis 107 and creates, from groove 69 to land 73 a gradual 0.025 mm clearance increase. That means that at the edge 690 separating the taper 72 from the groove 69, there is a clearance of approximately 0.0476 mm clearance between this edge 690 and the frusto-conical face 67 of the outflow portion 63 of the impeller drive shaft 55. The groove 69 spans an angular sector of approximately 20° about the longitudinal axis 107 computed from the edge 690 joining the taper 68 to the groove 69 up to the edge 691 joining the same groove 69 to the land 77. This angular sector of 20° includes the round of the edge 690 blending the groove 69 with the taper 68 and the round of the edge 691 blending the groove 69 with the land 77. The edges 692 that form the boundary between the groove 69 and the leading, annular rounded edge surface 460 are polished to create a round having a radius of at least 0.100 mm.

As a non-limitative example, from groove 70 to groove 71, the inner face 68 defines a taper 74 and a land 75. Taper 74 has a diameter that gradually decreases from groove 70 to land 75, and land 75 has a constant diameter. The land 75 spans an angular sector of approximately 20° about the longitudinal axis 107 and presents a clearance of approximately 0.0226 mm to the frusto-conical face 67 of the outflow portion 63 of the impeller drive shaft 55. The taper 74 spans an angular sector of approximately 80° about the longitudinal axis 107 and creates, from groove 70 to land 75 a gradual 0.025 mm clearance increase. That means that at the edge 700 separating the taper 74 from the groove 70, there is a clearance of approximately 0.0476 mm between this edge 700 and the frusto-conical face 67 of the outflow portion 63 of the impeller drive shaft 55. The groove 70 spans an angular sector of approximately 20° about the longitudinal axis 107 computed from the edge 700 joining the taper 74 to the groove 70 up to the edge 701 joining the same groove 70 to the land 73. This angular sector of 20° includes the round of the edge 700 blending the groove 70 with the taper 74 and the round of the edge 701 blending the groove 70 with the land 73. The edges 702 that form the boundary between the groove 70 and the leading, annular rounded edge surface 460 are polished to create a round having a radius of at least 0.100 mm.

As a non-limitative example, from groove 71 to groove 69, the inner face 68 defines a taper 76 and a land 77. Taper 76 has a diameter that gradually decreases from groove 71 to land 77, and land 77 has a constant diameter. The land 77 spans an angular sector of approximately 20° about the longitudinal axis 107 and presents a clearance of approximately 0.0226 mm to the frusto-conical face 67 of the outflow portion 63 of the impeller drive shaft 55. The taper 76 spans an angular sector of approximately 80° about the longitudinal axis 107 and creates, from groove 71 to land 77 a gradual 0.025 mm clearance increase. That means that at the edge 710 separating the taper 76 from the groove 71, there is a clearance of approximately 0.0476 mm clearance between this edge 710 and the frusto-conical face 67 of the outflow portion 63 of the impeller drive shaft 55. The groove 71 spans an angular sector of approximately 20° about the longitudinal axis 107 computed from the edge 710 joining the taper 76 to the groove 71 up to the edge 711 joining the same groove 71 to the land 75. This angular sector of 20° includes the round of the edge 710 blending the groove 71 with the taper 76 and the round of the edge 711 blending the groove 71 with the land 75. The edges 702 that form the boundary between the groove 71 and the leading, annular rounded edge surface 460 is polished to create a round having a radius of at least 0.100 mm.

In an example of construction, there are three blades such as 78 and 79 (FIG. 3) aligned with the three grooves 69, 70 and 71, respectively, to minimize blood flow perturbation. A number of blades and grooves different from 3 could obviously be used. Also, the blades such as 78 and 79 are connected to the grooves 69, 70 and 71, respectively, by diffusion bound.

In operation, the lands 73, 75 and 77 form a seat for the outflow frusto-conical face 67 of the outflow portion 63 of the impeller drive shaft 55. The grooves 69, 70 and 71 enable flow of blood between the faces 67 and 68. The hydrodynamic forces produced by rotation of the impeller drive shaft 55 will produce a thicker film of blood flowing between the frusto-conical face 67 of the outflow portion 63 of the impeller drive shaft 55 and the tapers 72, 74 and 76, and a thinner film of blood flowing between face 67 and the lands 73, 75 and 77 to thereby lubricate the resulting bearing (frusto-conical face 67 and frusto-conical bushing 46). The leading, annular rounded edge surface 460 at the edge of larger diameter will produce smooth flow of blood. Since blood flows through the gap between the frusto-conical faces 67 and 68, minimal hemolysis, thrombus and clot formation will be produced.

As a non-limitative example, the resulting outflow bearing (frusto-conical face 68 and frusto-conical bushing 46) present the following approximate dimensions and characteristics:
Larger diameter: 6 mm

Smaller diameter: 3 mm
Cone angle: 19.111°
Axial length: 4.1212 mm
Cone length: 4.3603 mm
Number of pads (taper and land): 3
Pad angle about the longitudinal axis: 100°
Groove angle about the longitudinal axis: 20°
Taper angle about the longitudinal axis: 80°
Taper gradual clearance increase: 0.025 mm
Land angle about the longitudinal axis: 20°

Land clearance: 0.0226 mm.

OUTFLOW 6807 RPM

| | Radial | Axial | Axial | Minimum | Power | Maximum |
|---|---|---|---|---|---|---|
| Clearance | Gap | Gap | Load | Film | Loss | Pressure |
| (mm) | (mm) | (mm) | (N) | (mm) | (Watt) | (Pa) |
| 0.0226 | 0.0241 | 0.0661 | −0.07 | 0.0226 | 0.0197 | 9643 |

with the above mentioned 0.8 N magnetic pull axially at 5 l/min against a pressure of 80 mmHg.

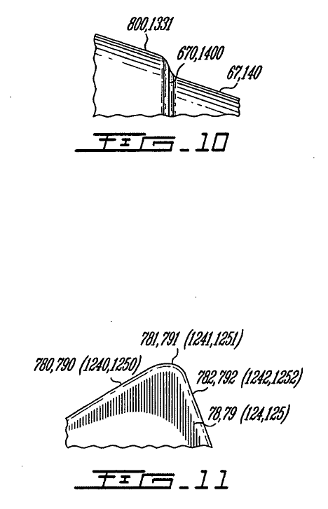

Referring to FIGS. 3 and 9, the impeller 56 comprises an annular, impeller blade structure 80.

Impeller blade structure 80 comprises an annular member 800 with an inner cylindrical surface 801 mounted on the outer cylindrical surface 630 of the outflow portion 63 of the impeller drive shaft 55 between an annular shoulder 631 of the outflow portion 63 and the frusto-conical face 67 of this outflow portion 63 of the impeller drive shaft 55. For example, the impeller blade structure 80 can be laser welded to the outflow portion 63 of the impeller drive shaft 55. As illustrated in FIG. 10, the annular junction 670 between the annular member 800 and the frusto-conical face 67 is positioned at the point of maximum slope.

The impeller blade structure 80 further comprises a set of impeller blades such as 78 and 79 evenly distributed inside the impeller housing 38 around a tapered frusto-conical face 802 of the annular member 800 of the impeller blade structure 80. It should be noted here that the annular radial-flow inlet 23 leads to the proximal end of the impeller blades such as 78 and 79 through a radial-flow inlet passage 231.

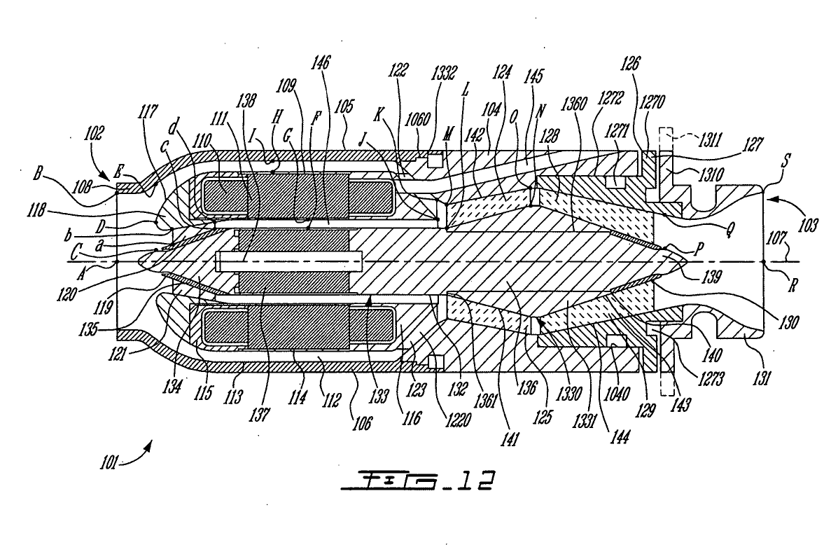

The shape (curvature and angulation) of the impeller blades such as 78 and 79 should be optimally designed in relation to pumping performance and other hydrodynamic considerations. In particular, the influence of the blade angulation on the level of shearing stresses, turbulence and cavitation responsible for red blood cell damage and increase of hemolysis rate must be carefully taken into consideration. To reduce the influence of blade angulation, FIG. 11 illustrates that each blade 78,79 has a full round radius at the top edge 780,790. Also, the radius that blends the top edge 781,791 with the trailing edge 782,792 forms a smooth surface to allow a better toolpath generation.

In the approach proposed by the illustrative intra-ventricular embodiment of the present invention, the mixed-flow blood pump 10 presents an enclosed-impeller mixed-flow configuration. The frusto-conical face 802 of the annular member 800 of the impeller blade structure 80, bearing the impeller blades such as 78 and 79 is tapered in the direction opposite to the direction of blood flow. This contributes to create the mixed-flow operation of the intra-ventricular mixed-flow blood pump 10. More specifically, this taper imparts to the blood flow both axial and radial components.

For housing the impeller blades, the impeller housing 38 comprises an inner frusto-conical surface 81 slightly less tapered in the direction opposite to the direction of blood flow than the face 802. To fit in the annular space defined between frusto-conical face 802 and the tapered surface 81, the width of the impeller blades such as 78 and 79 slightly and gradually decreases in the direction of blood flow. This also contributes to impart to the blood flow both axial and radial components.

The annular member 800 of the impeller blade structure also comprises an outer frusto-conical face 803 that is tapered in the direction of blood flow to fit within the outflow stator 45. The inner surface 83 of the outflow cannula 42 surrounding the outflow stator 45 is slightly less tapered in the direction of blood flow than the outer frusto-conical face 803. To fit in the annular space between the inner tapered surface 83 and the outer frusto-conical face 803, the radial blades such as 45 has a height that slightly and gradually increases in the direction of blood flow.

More specifically, the outer frusto-conical faces 802 and 803 and the inner surfaces 81 and 83 are shaped and dimensioned to keep the area through which blood flows constant from the leading end to the trailing end of the impeller blade structure 80.

The required electrical supply for the stator windings 26 is made through electrical wires extending through a conduit 84 itself extending from the cavity in which the stator windings 26 are installed through the annular portion 31, the radial blade 33, the hemispheric wall 32, and the hollow needle 20 to reach a controller and an energy source (both to be described hereinafter). Of course, this conduit 84 is sealed prior to implantation of the pump 10 within a human body.

Electric supply of the stator windings 26 will cause rotation of the impeller drive shaft 55 and therefore rotation of the set of impeller blades such as 78 and 79. More specifically, in the illustrative embodiment of FIG. 3, the mixed-flow blood pump 10 is actuated by means of a brushless DC (direct current) motor formed by the stator windings 26 housed in the cylindrical member 25 and the permanent magnet 64 embedded or housed in the impeller drive shaft 55. This brushless configuration presents the advantage of minimal wear. Two other interesting characteristics of brushless DC motors are high rotational speed and high torque.

As discussed in the following description, the cylindrical gap 86 between the outer surface of the impeller drive shaft 55 and the inner surface of the cylindrical member 25 must be sufficiently thick to produce sufficient blood flow in order to increase washout and prevent clot formation. However, increasing the thickness of the gap 86 decreases the efficiency of the magnetic coupling between the permanent magnet 64 and the stator windings 26. This requires an increase in current through the stator windings 26 to compensate for the decreased efficiency and to maintain the same characteristics in terms of impeller blade speed and blood volume throughput. Of course, increase in current leads to an increase in thermal loss from the stator windings 26; this thermal loss increases as the square of the current through the stator windings 26. As the temperature of the surface of the stator windings must remain at or below 40° C., the gap 86 must be sufficiently small to provide efficient magnetic coupling between the permanent magnet 64 and the stator windings 26.

Thermal performance is also improved given the proximate position of the stator windings 26 to the external surface 92 of the cylindrical member 25. Blood flow over the external surface 92 efficiently cools the stator windings 26. The flow of blood within the gap 86 also contributes in efficiently cooling the stator windings 26.

Axial spacing between the impeller blades such as 78 and 79 and the permanent magnet 64 along the impeller shaft 55 enables separate design of the motor and the impeller blades to obtain simultaneously both efficient coupling between the permanent magnet and the stator windings and sufficient pumping volume.

Rotation of the impeller blades such as 78 and 79 will impart pumping energy to the blood within the annular space between the outer frusto-conical face 802 and the inner frusto-conical surface 81. This will cause sucking of blood both through the annular radial-flow inlets 22 and 23. More specifically:
Blood flow enters the annular radial-flow inlet 22, is straightened out by the radial blades such as 33 and 34, fills the inflow chamber 85 with blood, is again straightened out by the radial blades such as 36 and 37 and is finally conducted toward the impeller blades such as 78 and 79 through the axial-flow inlet passage formed by the cylindrical gap 86 between the impeller drive shaft 55 and the inner surface of the cylindrical member 25; and
Blood flow enters the annular radial-flow inlet 23 and is conducted through the radial-flow inlet passage 231 where it is straightened out by the radial blades such as 39 and 40 to finally reach the impeller blades such as 78 and 79.

Blood flow then passes through the impeller blades such as 78 and 79, is straightened out by the outflow stator 45 and finally exits through the outflow cannula 42. As indicated in the foregoing description, the radial blades of the stationary outflow stator 45 are shaped and disposed to transform the rotational motion of the blood flow about the longitudinal axis 41 into a translational motion. Therefore, the stationary outflow stator 45 constitutes a blood flow straightener.

Still referring to FIG. 3, the cylindrical gap 86 separating the impeller shaft 55 and the inner surface of the cylindrical member 25 should be sufficiently thick to produce sufficient blood flow in order to increase washout and prevent clot formation. On the other hand, too large a gap 86 may either reduce the pump efficiency (by reducing the electromagnetic coupling) or result in higher hemolysis.

In the illustrative intra-ventricular embodiment of FIGS. 1 and 3, the volume of blood pumped through the second annular inlet 23 and the annular radial-flow inlet passage 231 is typically 3 liters/minute. This is higher than the volume of blood pumped through the first annular radial-flow inlet 22 and the axial-flow inlet passage formed by the cylindrical gap 86 which is typically 1 liter/minute. A number of benefits are associated with the higher volume of blood pumped through the second annular inlet 23. For example, installation of the mixed-flow blood pump 2 in the left ventricle 11 of a patient with the cannula 42 extending through the aortic valve generally interferes with proper operation of the aortic valve 15. Optimally, the aortic valve 15 should continue to function normally; however, in some cases, it has been observed that the aortic valve 15 ceases to function further until it remains closed around the cannula 42. Typically, blood would have the tendency to collect in the region close to the aortic valve and the cannula 42 which might lead to thrombus formation and other adverse effects. The increased volume of blood pumped through the second inlet 23 has the effect of creating blood flow in the region within the ventricle 11 delimited by the aortic valve 15 and the cannula 42, thus providing improved washout of this region and thereby reducing the negative effects of the malfunctioning aortic valve 15.

On the one hand, the volume of blood pumped through the second annular inlet 23 contributes to the radial-flow operation of the mixed-flow blood pump 10. On the other hand, the volume of blood pumped through the first annular inlet 22 and the cylindrical gap 86 (axial-flow inlet passage) contributes to the axial-flow operation of the mixed-flow blood pump 10.

The choice of materials for an implantable device such as the mixed-flow blood pump 10 is crucial and several properties of the available materials should be considered: strength, durability, hardness, elasticity, wear resistance, surface finish and biocompatibility. Biocompatibility is very important to minimise irritation, rejection and thrombogenesis. The interaction between the surface of the material and the biological tissues is very complex. In several cases, treatment of the surface with human proteins, certain drugs like heparin or other biocompatible material may considerably increase the biocompatibility and minimise thrombus formation (CBAS process, Carmeda AB).

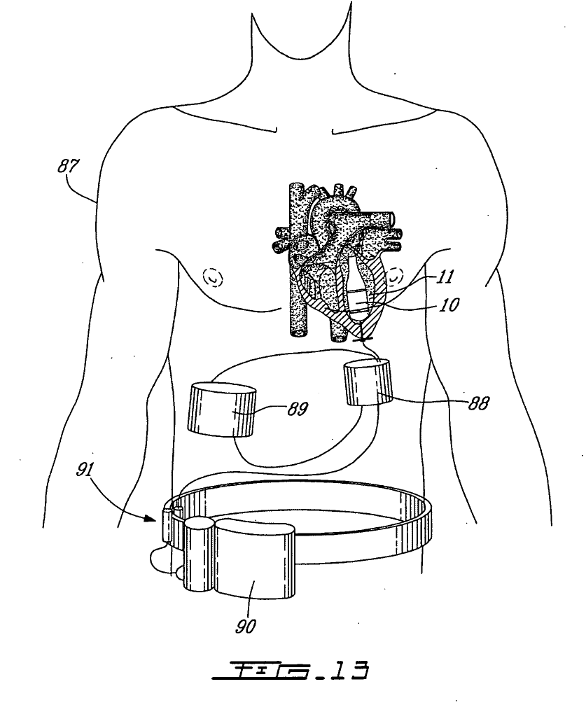

FIG. 12 illustrates an alternative, illustrative extra-ventricular embodiment 101 of mixed-flow blood pump according to the present invention. This illustrative embodiment 101 is adapted for use externally of the heart as a ventricle bypass/assist. This extra-ventricular blood pump 101 would typically be implanted above the diaphragm in the thorax and would be connected to the circulation system using standard vascular grafts, a first graft (not shown) being attached to the inflow end 102 of the pump and a second graft (not shown) being attached to the outflow end 103 of the pump.

Similar to the intra-ventricular mixed-flow blood pump 10 of FIG. 3, the extra-ventricular mixed-flow blood pump 101 as illustrated in FIG. 12 comprises a stationary housing structure 105 provided with an impeller housing 104.

The stationary housing structure 105 further comprises an outer cylindrical wall 106 centered on the longitudinal axis 107 of the extra-ventricular blood pump 101. The outer cylindrical wall 106 has a proximal end with a reduction of diameter 108 to receive the first graft and for connection to the patient's circulation system. The outer cylindrical wall 106 further comprises a distal end connected to the impeller housing 104. For example, the distal end of the cylindrical wall 106 and the proximal end of the impeller housing will have mutually mating cylindrical threaded portions at 1060 by means of which their are screwed on each other. A removable thread locking compound or a small radial hole drilled in the male threaded surface to include a plastic insert will then act as a thread locking device to prevent further rotation between the cylindrical wall 106 and the impeller housing 104. Finally, an annular groove 1041 will also be formed in the impeller housing 104 adjacent the threaded portions 1060 to receive and O-ring and form a tight seal between the cylindrical wall 106 and the impeller housing 104.

The stationary housing structure 105 of the intra-ventricular mixed-flow blood pump 10 comprises a hollow cylindrical member 109 containing the stator windings such as 110 and the associated magnetic cores such as 111. The hollow cylindrical member 109 is made of two mutually mating annular pieces 115 and 116 to enable insertion of the stator windings 110 and cores 111 within the hollow cylindrical member 109. For example, both the annular pieces 115 and 116 will be threaded to allow said annular pieces 115 and 116 to be screwed on each other. Further rotation of the annular pieces 115 and 116 on each other will then be prevented by any suitable means. Alternatively, the annular pieces 115 and 116 can be laser welded to each other.

As illustrated in FIG. 12, the hollow cylindrical member 109 is mounted within the outer cylindrical wall 106 coaxially therewith to form an annular axial-flow inlet passage 112 between the inner surface 113 of the outer cylindrical wall 106 and the outer surface 114 of the hollow cylindrical member 109.

The stationary housing structure 105 further comprises an inflow bushing mount 117 mounted on a proximal end of the cylindrical member 109. More specifically, the inflow bushing mount 117 comprises an annular portion 118 profiled to fit on the proximal end of the hollow cylindrical member 109 while defining with this cylindrical member 109 a smooth surface of the annular axial-flow inlet passage 112.

An inflow bushing 119 having the general configuration of a frustum of cone is mounted inside the annular portion 118 of the bushing mount 117 and is centered on the longitudinal axis 107 of the extra-ventricular blood pump 101. More specifically, the frusto-conical bushing 119 is mounted to the annular portion 118 through a series of radial blades such as 120 and 121 spread out evenly around the axis 107 of the extra-ventricular blood pump 101, more specifically around the frusto-conical bushing 119. As illustrated in FIG. 12, the frusto-conical bushing 119 has an end of larger diameter facing toward the inflow end of the extra-ventricular blood pump 101. Another function of the blades such as 120 and 121 is to straighten out the flow of blood passing between the frusto-conical bushing 119 and the annular portion 118 of the inflow bushing mount 117.

The distal end of the cylindrical member 109 is connected to the proximal end of the impeller housing 104 through a series of radial blades such as 122 and 123 spread out evenly around the longitudinal axis 107 to define an annular radial-flow inlet passage 1120 between the distal end of the annular flow passage 112 and the proximal end of the impeller blades such as 124 and 125. Another function of the radial blades such as 122 and 123 is to straighten out the flow of blood from the annular axial-flow inlet passage 112 and the annular radial-flow inlet passage 1220.

An outflow stator 126 comprises an annular member 127 mounted on the distal end of the impeller housing 104 through a flange 1270 and screws (not shown). An annular groove 1271 is provided on the outer cylindrical face 1272 of the annular member 127 to receive and O-ring (not shown) to ensure a tight seal between the outer cylindrical face 1272 of the annular member 127 and an inner cylindrical face 1040 of the impeller housing 104.

The outflow stator 126 also comprises a series of inner radial blades such as 128 and 129 spread out evenly around the longitudinal axis 107 and secured to the outflow stator 126 by diffusion bound. The radial blades such as 128 and 129 are configured to straighten out the flow of blood exiting the outflow stator 126. FIG. 4 illustrates an example of configuration of the radial blades such as 128 and 129 of the outflow stator 126. More specifically, the radial blades 128 and 129 are configured to straighten out the flow of blood exiting the outflow stator 126 and are fixedly secured to the inner face of the annular member 127.

An outflow bushing 130 having the general configuration of a frustum of cone is mounted inside the outflow stator 126 and is centered on the longitudinal axis 107. More specifically, the frusto-conical bushing 130 is mounted to the radial blades such as 128 and 129 of the outflow stator 126. Finally, the end of larger diameter of the frusto-conical bushing 130 is facing toward the inflow end of the extra-ventricular blood pump 101.

A blood diffuser 131 is mounted to the distal end of the outflow stator 126 through a flange 1310 through the same screws (not shown) that secure the flange 1270 of the outflow stator 126 to the impeller housing 104. The diameter of the flange 1310 may be extended (see for example 1311) to provide sewing holes therein for sewing the extra-ventricular mixed-flow blood pump 101 to the heart. nother annular groove 1273 of the annular member 127 receives an O-ring (not shown) to form a tight seal between the blood diffuser 131 and the outflow stator 126.

The function of the blood diffuser 131 is to increase the cross-sectional area of the pump outlet to the diameter of the second graft while minimising the shear stress on the blood cells. Since the velocity of the blood is inversely proportional to the cross-sectional area, the diffuser 131 will also reduce the velocity of the blood ejected from the extra-ventricular blood pump 101 to a velocity close to that of blood in the patient's circulation system. Of course, parameters such as the angle of opening and the length of the blood diffuser 131 could be adjusted at will to fit the mechanical characteristics of the extra-ventricular blood pump 101 in view of minimising the shear stress on the blood cells.

The extra-ventricular blood pump 101 also comprises a rotative impeller 133 provided with an impeller drive shaft 132 centered on the longitudinal axis 107. The impeller drive shaft 132 comprises an inflow end portion 134 formed with a frusto-conical face 135 structured to snugly fit into the frusto-conical bushing 119.

The inner face of the frusto-conical bushing 119 has the same structure and operation as the inner face 47 of the inflow bushing 35 of FIG. 3 described in detail with reference to FIG. 5.

Still referring to FIG. 12, the impeller drive shaft 132 also comprises an outflow portion 136 which, when assembled to the inflow end portion 134 defines a cavity in which a cylindrical permanent magnet 137 is inserted. The windings 110 and the permanent magnet 137 form an electric motor structure operative to set the impeller 133 into rotation; the magnetic field produced by the windings 110 is applied to the magnetic field produced by the permanent magnet 137 to produce a reaction that will set the impeller 133 into rotation.

An axial magnetic pull is produced by slightly, axially offsetting the permanent magnet 137 toward the inflow bushing 119 with respect of the magnetic windings 110. This will produce an axial magnetic pull of the order of, for example, 0.8 N toward the outflow bushing 130, i.e. in a direction opposite to an axial force produced on the impeller drive shaft 132 upon pumping blood.

An axial screw 138 passes through the magnet 137 and screws into both the inflow 134 and outflow 136 portions of the impeller drive shaft 132 to firmly secure these two drive shaft portions together.

The outflow portion 136 of the impeller drive shaft 132 comprises an outflow end 139 formed with a frusto-conical face 140 structured to snugly fit into the frusto-conical bushing 130.

Again, the inner face of the frusto-conical bushing 130 has the same structure and operation as the inner face 68 of the outflow bushing 46 of FIG. 3 described in detail with reference to FIG. 8.

Referring to FIG. 12, the impeller 133 comprises an annular, impeller blade structure 1330.

Impeller blade structure 1330 comprises an annular member 1331 with an inner cylindrical surface 1332 mounted on the outer cylindrical surface 1360 of the outflow portion 136 of the impeller drive shaft 132 between an annular shoulder 1361 of the outflow portion 136 and the frusto-conical face 140 of this outflow portion 136 of the impeller drive shaft 132. For example, the impeller blade structure 1330 can be laser welded to the outflow portion 136 of the impeller drive shaft 132.

Referring to FIG. 10, the annular junction 1400 between the annular member 1331 and the frusto-conical face 140 is positioned at the point of maximum slope.

The impeller blade structure 1330 further comprises a set of impeller blades such as 124 and 125 evenly distributed inside the impeller housing 104 around a tapered frusto-conical face 141 of the annular member 1331 of the impeller blade structure 1330. It should be noted here that the annular axial-flow 112 and radial-flow 1220 inlet passages lead to the proximal end of the impeller blades such as 124 and 125.

The shape (curvature and angulation) of the impeller blades such as 124 and 125 should be optimally designed in relation to pumping performance and other hydrodynamic considerations. In particular, the influence of the blade angulation on the level of shearing stresses, turbulence and cavitation responsible for red blood cell damage and increase of hemolysis rate must be carefully taken into consideration. To reduce the influence of blade angulation, FIG. 11 illustrates that each blade 124,125 has a full round radius at the top edge 1240,1250. Also, the radius that blends the top edge 1241,1251 with the trailing edge 1242,1252 forms a smooth surface to allow a better toolpath generation.

In the approach proposed by the illustrative extra-ventricular embodiment of the present invention, the mixed-flow blood pump 101 presents an enclosed-impeller mixed-flow configuration. The frusto-conical face 141 of the annular member 1331 of the impeller blade structure 1330, bearing the impeller blades such as 124 and 125 is tapered in the direction opposite to the direction of blood flow. This contributes to create the mixed-flow operation of the intra-ventricular mixed-flow blood pump 101. More specifically, this taper imparts to the blood flow both axial and radial components.

For housing the impeller blades, the impeller housing 104 comprises an inner frusto-conical surface 142 slightly less tapered in the direction opposite to the direction of blood flow than the face 141. To fit in the annular space defined between frusto-conical face 141 and the tapered surface 142, the width of the impeller blades such as 124 and 125 slightly and gradually decreases in the direction of blood flow. This also contributes to impart to the blood flow both axial and radial components.

The annular member 1331 of the impeller blade structure 1330 also comprises an outer frusto-conical face 143 that is tapered in the direction of blood flow to fit within the outflow stator 126. The inner surface 144 of the annular member 127 is slightly less tapered in the direction of blood flow than the frusto-conical face 143. To fit in the annular space between the inner tapered surface 144 and the frusto-conical face 143, the radial blades such as 128 and 129 have a height that slightly and gradually increases in the direction of blood flow.

The required electrical supply for the stator windings 110 is made through electrical wires extending through a conduit 145 itself extending from the cavity in which the stator windings 110 are installed through the annular piece 116, the radial blade 122, and the impeller housing 104 to reach a controller and an energy source (both to be described hereinafter). Of course, this conduit 145 is sealed prior to implantation of the pump 101 within a human body.

Electric supply of the stator windings 110 will cause rotation of the impeller drive shaft 132 and therefore rotation of the set of impeller blades such as 124 and 125. More specifically, in the illustrative embodiment of FIG. 12, the mixed-flow blood pump 101 is actuated by means of a brushless DC (direct current) motor formed by the stator windings 110 housed in the cylindrical member 109 and the cylindrical permanent magnet 137 embedded or housed in the impeller drive shaft 55. This brushless configuration presents the advantage of minimal wear. Two other interesting characteristics of brushless DC motors are high rotational speed and high torque.

Both the annular axial-flow passage 112 and the cylindrical gap 146 between the outer surface of the impeller drive shaft 132 and the inner surface of the cylindrical member 109 must be both sufficiently thick to produce sufficient blood flow in order to increase washout and prevent clot formation. However, increasing the thickness of the gap 146 decreases the efficiency of the magnetic coupling between the permanent magnet 137 and the stator windings 110. This requires an increase in current through the stator windings 110 to compensate for the decreased efficiency and to maintain the same characteristics in terms of impeller blade speed and blood volume throughput. Of course, increase in current leads to an increase in thermal loss from the stator windings 110; this thermal loss increases as the square of the current through the stator windings 110. As the temperature of the surface of the stator windings must remain at or below 40° C., the gap 146 must be sufficiently small to provide efficient magnetic coupling between the permanent magnet 137 and the stator windings 110.

Thermal performance is also improved given the proximate position of the stator windings 110 to the annular axial-flow passage 112 and the annular axial-flow inlet passage formed by the cylindrical gap 146. Blood flow through these annular axial-flow passages efficiently cools the stator windings 110.

Axial spacing between the impeller blades such as 124 and 125 and the permanent magnet 137 along the impeller shaft enables separate design of the motor and the impeller blades to obtain simultaneously both efficient coupling between the permanent magnet and the stator windings and sufficient pumping volume.

Rotation of the impeller blades such as 124 and 125 will impart pumping energy to the blood within the annular space between the frusto-conical annular face 141 and the tapered inner surface 142 of the impeller housing 104. This will cause sucking of blood both through the annular axial-flow inlet passage 112 and the annular axial-flow inlet passage formed by the cylindrical gap 146. More specifically:
Blood flows through the annular axial-flow inlet passage 112 and the radial-flow inlet passage 1220 and is straightened out by the radial blades such as 122 and 123 to reach the impeller blades such as 124 and 125; and
Blood flow is straightened out by the radial blades such as 120 and 121 to reach the impeller blades such as 124 and 125 through the axial-flow inlet passage formed by the cylindrical gap 146.

Blood flow then passes through the impeller blades such as 124 and 125, is straightened out by the outflow stator blades such as 128 and 129 and finally exits through the blood diffuser 131. The radial blades such as 128 and 129 of the stationary outflow stator 126 are shaped and disposed to transform the rotational motion of the blood flow about the longitudinal axis 107 into a translational motion. Therefore, the stationary outflow stator 126 constitutes a blood flow straightener.

Again, the choice of materials for an implantable device such as the mixed-flow blood pump 101 is crucial and several properties of the available materials should be considered: strength, durability, hardness, elasticity, wear resistance, surface finish and biocompatibility. Biocompatibility is very important to minimise irritation, rejection and thrombogenesis. The interaction between the surface of the material and the biological tissues is very complex. In several cases, treatment of the surface with human proteins, certain drugs like heparin or other biocompatible material may considerably increase the biocompatibility and minimise thrombus formation.

The following features of the mixed-flow blood pump 10, 101 have been designed to (a) minimize flow energy losses and (b) eliminate stagnation zones leading to thrombus formation:
The outer 231,112 and inner 86,146 inlet passages of the mixed-flow blood pump 10,101 divide the blood flow according to a ratio of 3:1, respectively, which corresponds to the same ratio between the areas between these two inlet passages. This presents the advantage of reducing by 75% the shear stress induced by the rotation of the impeller shaft 56,136 to the blood flowing through the inner inlet passage 86,146, while also reducing the corresponding energy losses (see above point (a)). Contrary to a pump having only one inner inlet passage, the mixed-flow blood pump is therefore less traumatic.
The outer inlet passage 112 meets with the radial inlet passage 231,1220 to supply the impeller with blood. The cross section of the radial-flow inlet passage 231,1220 is designed equal to the cross section of the outer inlet passage to prevent an acceleration of the flow (see above point (a)) or a sudden reduction of the speed of blood flow to provoke pump's free wheeling (see above point (b)).
The inner axial-flow inlet passage 86,146 is the confluent of the radial-flow inlet passage 231,1220 and completes the supply of blood to the impeller. Since the ratio of these two flows are equal to the ratio of the areas of the axial-flow and radial-flow inlet passages at the junction of these inlet passages, the blood flows join each other at equal speeds to avoid production of blood shearing zones (see above point (a)) or interruption of the blood flow (see above point (b)). Indeed, when two jets join each other at unequal speeds, a whirling motion flow results at the junction to increase the losses (see above point (a)).
The cross sectional area of the annular passage at the inlet of the set of impeller blades (78,79),(124,125) is made by purpose slightly smaller than the cross sectional area of the annular passage at the junction between the outer radial-flow 231,1020 and inner axial-flow 86,146 inlet passages. In this small zone, a slight acceleration of the blood flow is produced which slightly reduces the pressure to improve sucking of blood within the impeller blades (78,79),(124,125) over the entire operating range of the pump. However, too high an acceleration would provoke an interruption in the blood flow and therefore would be detrimental to the performance of the mixed-flow blood pump 10,101. In the present illustrative design, a beneficial trade-off was reached to improve the performance of the mixed-flow blood pump.
The cross sectional area of the annular passage from the inlet to the outlet of the set of impeller blades (78,79),(124,125) is constant. In this manner, the flow is not axially accelerated within this passage whereby the energy losses are minimized. Geometrically, the design of radial blades is allowed while the transfer of pressure energy and whirling kinetic energy is favoured.
The cross sectional area of the annular passage from the inlet to the outlet of the set of outflow stator blades 450, 128, 129 is constant and equal to the cross sectional area of the annular passage from the inlet to the outlet of the set of impeller blades (78,79),(124,125). This will prevent pump free-wheeling (see above point (b)) and minimize losses (see above point (a)) for a better efficiency in this important portion of the mixed-flow blood pump 10, 101 whose role is to convert the whirling kinetic energy into pressure energy. However, it should be noted that the cross sectional area of the annular passage at the outlet of the set of outflow stator blades is slightly smaller than the cross sectional area of this annular passage at the inlet of the set of outflow stator blades due to the presence of the outflow bearing 46, 130. By provoking in this manner a slight acceleration of the blood flow, we obtain good cleaning of the inner space of the outflow bearing. This approach is used for the inflow bearing 35,119 as well.

Examples of radial dimensions and cross sectional areas for the extra-ventricular mixed-flow blood pump 101 of FIG. 12 are given in the following Table. Intra-ventricular mixed-flow blood pump 10 of FIG. 3 has similar dimensions.

| Location (see FIG. 12) | Radius (mm) | Cross-sectional area (mm²) |
|---|---|---|
| A | 0 | 64 |
| (50 microns) | | |
| B | 8 | |
| C | 1.31 | 19.27 |
| D | 4.581 | 54.74 |
| approximately | | |
| E | 8.702 | |
| approximately | | |
| a | 2.048 | 10.96 |
| b | 3.893 | |
| c | 3.850 | 7.30 |
| d | 4.703 | |
| F | 4 | 9.00 |
| G | 5 | |
| H | 10.5 | 27.81 |
| I | 11.75 | |
| J | 5 | 27.81 |
| K | 7.267 | |
| L | 4 | 30.24 |
| M | 6.8 | |
| N | 6.62 | 30.24 |
| O | 8.606 | |
| P | 1.475 | 28.83 |
| Q | 5.5685 | |
| R | 0 | 64 |
| (50 microns) | | |
| S | 8 | |

FIG. 13 schematically illustrates an embodiment of implantable VAD system including an axial-flow blood pump 10. The VAD system is composed of four main parts:
the axial-flow blood pump 10 implanted in the left ventricle 11 of the patient 87;
an internal controller 88;
two energy sources, namely an internal rechargeable battery 89 and an external rechargeable battery 90; and
a Transcutaneous Energy and Information Transmission (TEIT) system 91.

VAD and TEIT Systems are well known in the art and will not be further discussed in the present specification.

To conclude, ventricular assist devices (VADs) are now being used worldwide and their utilisation is becoming more and more accepted as a solution to treat end stage heart failure. It is generally accepted that VADs extend life of patients while improving quality of life of these patients. A poll, made with patients who received VADs, concerning their quality of life revealed that these patients would have preferred a heart transplant but prefer their situation than having to be on dialyses.

It is also now being accepted that VAD is becoming a cost effective solution considering the fact that patients are discharged from the hospitals more rapidly and may return to normal life occupations. In the United States, several insurance companies are now reimbursing the implantation of VADs.

Finally, the mixed-flow blood pump 10 according to the illustrative embodiments of the present invention provides an excellent bridge to heart transplant and aims at long term implant. The new proposed mixed-flow blood pump 10 should answer most of the remaining problems and limitations of the prior axial-flow blood pumps, especially those related to hemolysis. Hemolysis is the tearing of red blood cells, which empties the content of the cells in the blood stream resulting in free haemoglobin; the normal level of plasma free haemoglobin is around 10 mg/dl. A blood pump with a normalised index of hemolysis (NIH) of 0.005 g/100 litres and lower is considered to be almost athromatic for red blood cells. A NIH of about 0.05 g/100 litres could be tolerated. A NIH situated between 0.005 g/100 litres to 0.05 g/100 litres can therefore be envisaged for a VAD. Of course, a NIH as close to 0.005 g/100 litres as possible is desirable.

Although the present invention has been described hereinabove by way of illustrative embodiments thereof, these embodiments can be modified at will, within the scope of the appended claims, without departing from the spirit and nature of the present invention.

1. A blood pump comprising:
a stationary housing structure having a proximal end and a distal end, said stationary housing structure being substantially symmetrical about a longitudinal axis;
a rotative impeller mounted within the stationary housing structure to circulate blood in a blood flow direction extending from said proximal end to said distal end;
a first inlet provided near said proximal end; said first inlet leading to a first passage;
a second inlet leading to a second passage defining an acute angle with the longitudinal axis; and
said first and second passages joining into a common passage between said second inlet and said rotative impeller;
whereby when said rotative impeller is activated a first predetermined volume of blood flows in said stationary housing structure through said first inlet and a second predetermined volume of blood flows in said stationary housing structure through said second inlet; said second predetermined volume being greater than said first predetermined volume.

2. A blood pump as recited in claim 1, wherein said second inlet is located between the first inlet and the rotative impeller.

3. A blood pump as recited in claim 1, wherein a substantial portion of said stationary housing structure is of a generally cylindrical shape.

4. A blood pump as recited in claim 1, wherein said second inlet includes a plurality of apertures radially spread about said longitudinal axis.

5. A blood pump as recited in claim 1, wherein said first passage is generally parallel to said longitudinal axis.

6. A blood pump as recited in claim 1, wherein said first passage is defined by a generally annular gap between said rotative impeller and said stationary housing structure.

7. A blood pump as recited in claim 1, further comprising blood straightening elements provided in the vicinity of said first inlet to straighten a blood flow from said first inlet.

8. A blood pump as recited in claim 7, wherein said first blood straightening elements include radial blades.

9. A blood pump as recited in claim 1, comprising blood straightening elements provided in the second passage to straighten a blood flow from said second inlet.

10. A blood pump as recited in claim 9, wherein said second blood straightening elements include radial blades.

11. A blood pump as recited in claim 1, wherein:
blood entering the blood pump by said first inlet defines a first blood flow having a first pressure;
blood entering the blood pump by said second inlet defines a second blood flow having a second pressure; and
said first and second passage are so configured and sized that said first and second pressures are similar.

12. A blood pump as recited in claim 1, wherein said second predetermined volume of blood entering said stationary housing structure is about three times greater than said first predetermined volume of blood entering said stationary housing structure.

13. A blood pump as recited in claim 1, wherein said rotary impeller is magnetically driven.

14. A blood pump as recited in claim 1, wherein said common passage between said second inlet and said rotative impeller tapers in the blood flow direction to produce a slight acceleration of the blood flow thereby slightly reducing the pressure to improve sucking of blood within the rotative impeller.

15. A blood pump as recited in claim 1, wherein a cross-sectional area of said common passage at the junction of said first and second passage is slightly larger than a cross-sectional area of said common passage at said rotative impeller to produce a slight acceleration of the blood flow thereby slightly reducing the pressure to improve sucking of blood within the rotative impeller.
